(19) United States
(12) Patent Application Publication
(10) Pub. No.: US 2026/0228762 A1
(43) Pub. Date: Aug. 6, 2026

(54) SYSTEMS AND METHODS FOR GENERATING AND USING SIMULATED CUSTOMER PROFILES

(71) Applicant: JPMORGAN CHASE BANK, N.A., New York, NY (US)

(72) Inventors: Sandeep KOLLA, McKinney, TX (US); Surendra KATIKAREDDY, Celina, TX (US)

(21) Appl. No.: 19/629,944

(22) Filed: Mar. 26, 2026

Related U.S. Application Data
(60) Provisional application No. 63/178,177, filed on Apr. 22, 2021.

(51) Int. Cl. G06Q 30/0201; G06F 9/455

(52) U.S. Cl. CPC G06Q 30/02011; G06F 9/455

(57) ABSTRACT

A method may include: receiving, by a computer program executed by a backend electronic device, a communication a customer regarding a mobile application; retrieving, by the computer program, customer information associated with the customer; identifying, by the computer program, a potential issue with the mobile application based on the customer information; generating, by the computer program, mock data for the customer; generating, by the computer program, a simulated customer profile from the mock data and the customer information; simulating, by the computer program, execution of the mobile application using a mobile application simulator for the mobile application using the simulated customer profile; troubleshooting, by the computer program, the potential issue using the mobile application simulator; and sending, by the computer program, a control signal to the mobile application to address the potential issue.

### RELATED APPLICATIONS

This application claims priority to, and the benefit of, U.S. Provisional Patent Application Ser. No. 63/778,177, filed Mar. 26, 2025, the disclosure of which is hereby incorporated, by reference, in its entirety.

### BACKGROUND OF THE INVENTION

### 1. Field of the Invention

Embodiments relate to systems and methods for generating and using simulated customer profiles.

### 2. Description of the Related Art

Many organizations provide computer applications for their customers to use. For example, online retailers provide mobile applications for customers to place, cancel, track, and return orders; financial institutions provide mobile applications for customers to check balances, transfer funds, make payments, view transactions, etc. Within the mobile applications, different customers may be provided with different features, and customers may also have the opportunity to customize the way that information is presented. In view of the differences in the way that customers may be presented with information in the mobile application, a customer service representative assisting a customer may have difficulty knowing what the customer is seeing at a particular time, or recreating a troublesome scenario.

### SUMMARY OF THE INVENTION

Systems and methods for generating and using simulated customer profiles are disclosed. In an embodiment, a method may include: receiving, by a computer program executed by a backend electronic device, a communication a customer regarding a mobile application; retrieving, by the computer program, customer information associated with the customer; identifying, by the computer program, a potential issue with the mobile application based on the customer information; generating, by the computer program, mock data for the customer; generating, by the computer program, a simulated customer profile from the mock data and the customer information; simulating, by the computer program, execution of the mobile application using a mobile application simulator for the mobile application using the simulated customer profile; troubleshooting, by the computer program, the potential issue using the mobile application simulator; and sending, by the computer program, a control signal to the mobile application to address the potential issue.

In one embodiment, the customer information may include account information, mobile application feature information, and customer preferences for the mobile application.

In one embodiment, the potential issue with the mobile application may be identified using a rules based engine.

In one embodiment, the potential issue with the mobile application may be identified using a database of known issues.

In one embodiment, the potential issue with the mobile application may be identified using a machine learning model trained on historical support cases.

In one embodiment, the mock data may be generated based on predefined templates for the potential issue.

In one embodiment, the mock data may include plausible merchant names, plausible transaction amounts, and/or plausible error messages.

In one embodiment, the mock data may be generated using a large language models.

In one embodiment, the control signal causes the mobile application to turn a feature on or off.

In one embodiment, the control signal causes the mobile application to display a page.

According to another embodiment, a non-transitory computer readable storage medium may include instructions stored thereon, which when read and executed by one or more computer processors, cause the one or more computer processors to perform steps including: receiving a communication a customer regarding a mobile application; retrieving customer information associated with the customer; identifying a potential issue with the mobile application based on the customer information; generating mock data for the customer; generating a simulated customer profile from the mock data and the customer information; simulating execution of the mobile application using a mobile application simulator for the mobile application using the simulated customer profile; troubleshooting the potential issue using the mobile application simulator; and sending a control signal to the mobile application to address the potential issue.

In one embodiment, the customer information may include account information, mobile application feature information, and customer preferences for the mobile application.

In one embodiment, the potential issue with the mobile application may be identified using a rules based engine.

In one embodiment, the potential issue with the mobile application may be identified using a database of known issues.

In one embodiment, the potential issue with the mobile application may be identified using a machine learning model trained on historical support cases.

In one embodiment, the mock data may be generated based on predefined templates for the potential issue.

In one embodiment, the mock data may include plausible merchant names, plausible transaction amounts, and/or plausible error messages.

In one embodiment, the mock data may be generated using a large language models.

In one embodiment, the control signal causes the mobile application to turn a feature on or off.

In one embodiment, the control signal causes the mobile application to display a page.

### BRIEF DESCRIPTION OF THE DRAWINGS

For a more complete understanding of the present invention, the objects and advantages thereof, reference is now made to the following descriptions taken in connection with the accompanying drawings in which:

FIG. 1 illustrates a system for generating and using simulated customer profiles according to an embodiment;

FIG. 2 depicts a method for generating and using simulated customer profiles according to an embodiment;

FIG. 3 depicts an exemplary computing system for implementing aspects of the present disclosure.

### DETAILED DESCRIPTION OF PREFERRED EMBODIMENTS

Systems and methods for generating and using simulated customer profiles are disclosed. For example, embodiments may create a simulated customer profile that does not include personal or sensitive information, and can be used to simulate the customer's experience with a mobile application. For example, in a banking application, the customer profile may include account types (e.g., checking, savings, credit card, mortgage, loans, etc.), features that are available to the customer (e.g., application features that may not be available to all customers), and customer application preferences (e.g., the order in which information is presented in the mobile application). The simulated customer profile may also identify any error messages that the customer may have received. Using this simulated customer profile, a customer service representative may execute a simulated mobile application with the simulated customer profile, and may attempt to recreate any errors that the customer has received.

In another embodiment, the customer service agent may assist the customer in navigating different screens and flows in the mobile application, and may provide the customer with step-by-step instructions for reaching a desired screen or data point.

Embodiments may also facilitate experimentation using different simulated customer profiles to determine how the mobile application will react or operate.

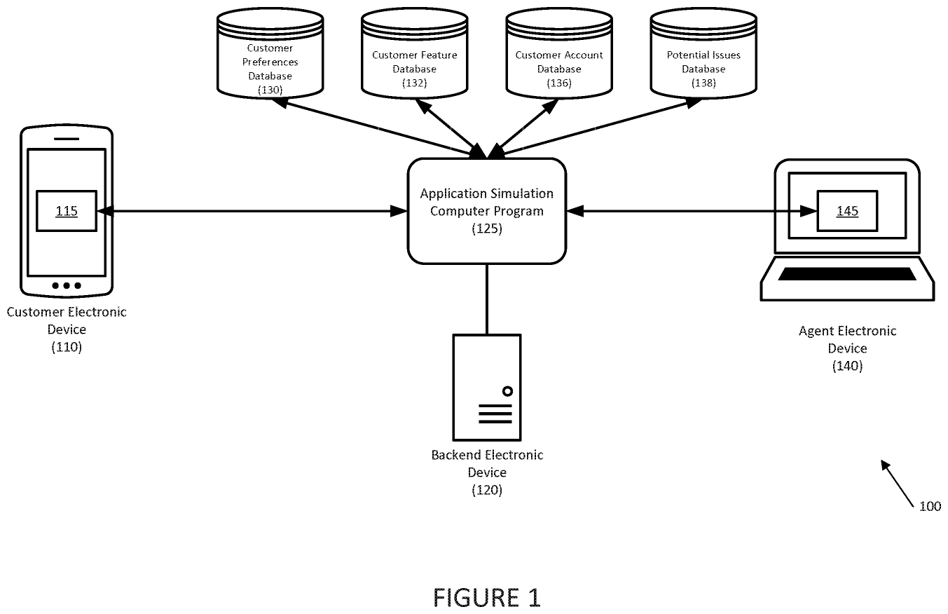

Referring to FIG. 1, a system for generating and using simulated customer profiles is disclosed. System 100 may include customer electronic device 110, such as a smart phone, tablet computer, smart watch, etc. In general, customer electronic device 110 may execute mobile application 115 that may be provided by an entity or organization.

System 100 may further include backend electronic device 120, which may be a server (e.g., physical and/or cloud-based), a computer, etc. Backend electronic device 120 may execute application simulation computer program 125, which may generate a simulated customer profile and may execute it on a simulated version of mobile application 115.

Application simulation computer program may generate the simulated customer profile using data from customer preferences database 130, customer feature database 132, and/or customer account database 136. Customer preferences database 130 may maintain customer preferences for the manner in which information is displayed by mobile application 115. For example, customer preferences may specify the customer's preference for user configurable elements of mobile application 115, such as order in which information is presented, color schemes, etc.

Customer feature database 132 may identify features that are available to the customer in mobile application 115. For example, certain features may be turned on or off, may be located differently for different customers, etc.

Customer feature database 132 may also identify any errors that the customer has been presented with by mobile application 115.

Customer account database 136 may identify account(s) that the customer may have with the organization. Customer account database 136 may only identify a type of account, but may not provide any account information (e.g., account numbers, balances, transactions, etc.).

Potential issues database 138 may maintain a list of potential issues that the customer may encounter with mobile application 115, such as failed transaction, disabled features, incorrect balance display. etc. Potential issues database may include templates that may guide the generation of mock data for each issue.

System 100 may further include agent electronic device 140, which may be a computer, a terminal, etc. that may interface with application simulation computer program 125. In an embodiment, agent electronic device 140 may execute agent computer program 145, which may allow an agent to simulate the execution of mobile application 115 by the customer by using the customer simulated profile for the customer.

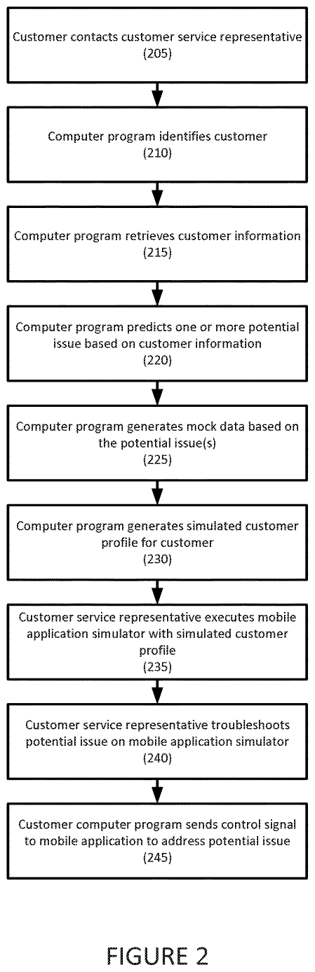

Referring to FIG. 2, a method for generating and using simulated customer profiles is disclosed according to an embodiment.

In step 205, a customer may contact a customer service representative. The customer may contact the customer service representative about an issue with a mobile application, may request assistance navigating in the mobile application (e.g., locating a screen or feature), etc.

In step 210, the computer program may identify the customer. The identification may include authenticating the customer.

As a result of the identification, a unique identifier for the customer may be identified.

In step 215, the computer program may retrieve customer information for the customer identifier. For example, the computer program may retrieve account information (e.g., types of accounts, balances, etc.), application feature information (e.g., application features that are turned on or off for the customer), and customer preferences for the mobile application.

In step 220, the computer program may identify one or more potential issue based on the customer information retrieved. In an embodiment, the computer program may identify potential issues experienced by the customer based on the retrieved customer information. This identification may be performed using a rules-based engine, a database of known issues, or machine learning models trained on historical support cases. For example, the computer program may compare the customer's recent activity, error logs, and feature usage against a database of common issues to flag likely problems. In other embodiments, a machine learning classifier may analyze patterns in the customer's data to predict or rank potential issues. The identification process may consider factors such as recent failed transactions, disabled features, unusual account activity, or error messages received by the customer.

Illustrative examples are as follows. If a customer's account shows a recent failed transaction and an associated error code, the computer program may identify “transaction failure” as a potential issue. If a feature is disabled for the customer but is commonly enabled for similar profiles, the computer program may flag “feature access issue” as a potential issue.

In step 225, once one or more potential issues are identified, the computer program may generate mock data corresponding to those issues. The mock data is designed to simulate the customer's experience without exposing any personal or sensitive information. Mock data generation may be guided by:
predefined templates for common issues (e.g., failed transaction, disabled features, incorrect balance display, etc.); randomized but realistic synthetic data generation, such as generating plausible merchant names, plausible transaction amounts, or plausible error messages; the use of generative artificial intelligence models, such as large language models (LLMs), to create realistic but anonymized data that reflects the structure and content of actual customer data.

Examples of mock data may include account balances (with relative values, not actual numbers), transaction histories (with randomized merchants, dates, and amounts), feature availability flags (on/off), error messages or codes, application preferences (e.g., color schemes, dashboard order), etc.

Illustrative examples are as follows. If the identified potential issue is a failed transaction, the system may generate a mock transaction with a merchant name such as “SampleMart” and an amount of “$45.67,” along with an error message such as “Transaction failed: insufficient funds.” If the potential issue is a disabled feature, the mock data may indicate that the “mobile deposit” feature is turned off.

In step 230, using the mock data, the application feature information, and the customer preferences, the computer program may generate a simulated customer profile for the customer. The simulated customer profile may be constructed by combining the generated mock data, feature availability information, and customer preferences into a data structure that mirrors the actual customer profile used by the mobile application. The simulated customer profile contains all relevant (but non-sensitive) data needed to reproduce the customer's application state and experience.

In an embodiment, the simulated customer profile may be represented as a structured data object, such as a JSON object, a database record, or another suitable format. The simulated customer profile may then be loaded into a simulation environment to reproduce the customer's experience for troubleshooting or support purposes.

An illustrative example of a simulate profile (in JSON) is provided below:

```
{
  “account_types”: [“checking”, “savings”],
  “balances”: {“checking”: “$1,234”, “savings”: “$5,678”},
  “features_enabled”: [“mobile_deposit”, “bill_pay”],
  “recent_transactions”: [
    {“date”: “2026-03-01”, “merchant”: “SampleMart”, “amount”:
“$45.67”},
    {“date”: “2026-03-03”, “merchant”: “DemoStore”, “amount”:
“$123.45”}
  ],
  “preferences”: {“theme”: “dark”, “dashboard_order”:
[“balances”, “transactions”, “offers”]},
  “error_messages”: [“Transaction failed: insufficient funds”]
}
```

In step 235, the customer service representative may execute a mobile application simulator using the simulated customer profile. This may allow a customer service representative to view and interact with the application as the customer would, but without exposing any sensitive information.

The mobile application simulator simulates the execution of the mobile application by the customer, and results in the customer service representative being presented with the same screen, features, etc. that the customer is presented with without the personal or sensitive information.

In step 240, the customer service representative may troubleshoot the potential issue the customer is having, or may assist the customer in locating a feature, screen, etc. by navigating the mobile application with the customer. For example, the customer service representative may provide a step-by-step walkthrough for the customer.

In step 245, the computer program may send a control signal to the mobile application to address the potential issue. For example, the control signal may cause the mobile application to turn on or to turn off a feature, it may cause the mobile application to display a setting, it may cause the mobile application to display a certain page, it may allow the customer service representative to remotely control the mobile application, etc.

Other actions may include feature or configuration correction actions, such as toggling a feature flag to intended state based on policy/entitlements, publishing feature flags to customer current session and verify actions resulted in remediation, rolling back a customer-specific configuration to a last-known-good profile snapshot, etc.

Examples of observability actions may include generating and attaching a machine-readable diagnostics bundle (e.g., an error code, a trace ID, a context signature, etc.), emitting structured telemetry event for incident correlation and future model/rule improvement, etc.

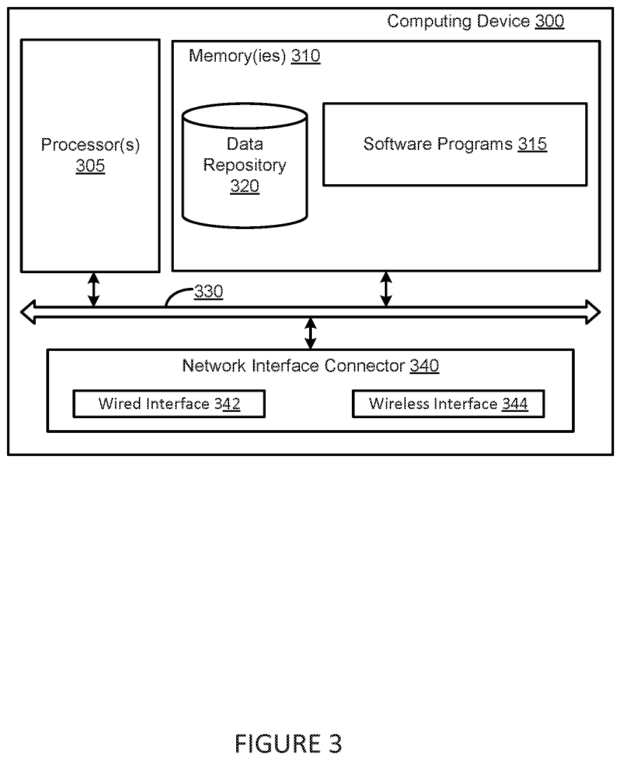

FIG. 3 depicts an exemplary computing system for implementing aspects of the present disclosure. FIG. 3 depicts exemplary computing device 300. Computing device 300 may represent the system components described herein. Computing device 300 may include processor 305 that may be coupled to memory 310. Memory 310 may include volatile memory. Processor 305 may execute computer-executable program code stored in memory 310, such as software programs 315. Software programs 315 may include one or more of the logical steps disclosed herein as a programmatic instruction, which may be executed by processor 305. Memory 310 may also include data repository 320, which may be nonvolatile memory for data persistence. Processor 305 and memory 310 may be coupled by bus 330. Bus 330 may also be coupled to one or more network interface connectors 340, such as wired network interface 342 or wireless network interface 344. Computing device 300 may also have user interface components, such as a screen for displaying graphical user interfaces and receiving input from the user, a mouse, a keyboard and/or other input/output components (not shown).

Hereinafter, general aspects of implementation of the systems and methods of embodiments will be described.

Embodiments of the system or portions of the system may be in the form of a “processing machine,” such as a general-purpose computer, for example. As used herein, the term “processing machine” is to be understood to include at least one processor that uses at least one memory. The at least one memory stores a set of instructions. The instructions may be either permanently or temporarily stored in the memory or memories of the processing machine. The processor executes the instructions that are stored in the memory or memories in order to process data. The set of instructions may include various instructions that perform a particular task or tasks, such as those tasks described above. Such a set of instructions for performing a particular task may be characterized as a program, software program, or simply software.

In an embodiment, the processing machine may be a specialized processor.

In an embodiment, the processing machine may be a cloud-based processing machine, a physical processing machine, or combinations thereof.

As noted above, the processing machine executes the instructions that are stored in the memory or memories to process data. This processing of data may be in response to commands by a user or users of the processing machine, in response to previous processing, in response to a request by another processing machine and/or any other input, for example.

As noted above, the processing machine used to implement embodiments may be a general-purpose computer. However, the processing machine described above may also utilize any of a wide variety of other technologies including a special purpose computer, a computer system including, for example, a microcomputer, mini-computer or mainframe, a programmed microprocessor, a micro-controller, a peripheral integrated circuit element, a CSIC (Customer Specific Integrated Circuit) or ASIC (Application Specific Integrated Circuit) or other integrated circuit, a logic circuit, a digital signal processor, a programmable logic device such as a FPGA (Field-Programmable Gate Array), PLD (Programmable Logic Device), PLA (Programmable Logic Array), or PAL (Programmable Array Logic), or any other device or arrangement of devices that is capable of implementing the steps of the processes disclosed herein.

The processing machine used to implement embodiments may utilize a suitable operating system.

It is appreciated that in order to practice the method of the embodiments as described above, it is not necessary that the processors and/or the memories of the processing machine be physically located in the same geographical place. That is, each of the processors and the memories used by the processing machine may be located in geographically distinct locations and connected so as to communicate in any suitable manner. Additionally, it is appreciated that each of the processor and/or the memory may be composed of different physical pieces of equipment. Accordingly, it is not necessary that the processor be one single piece of equipment in one location and that the memory be another single piece of equipment in another location. That is, it is contemplated that the processor may be two pieces of equipment in two different physical locations. The two distinct pieces of equipment may be connected in any suitable manner. Additionally, the memory may include two or more portions of memory in two or more physical locations.

To explain further, processing, as described above, is performed by various components and various memories. However, it is appreciated that the processing performed by two distinct components as described above, in accordance with a further embodiment, may be performed by a single component. Further, the processing performed by one distinct component as described above may be performed by two distinct components.

In a similar manner, the memory storage performed by two distinct memory portions as described above, in accordance with a further embodiment, may be performed by a single memory portion. Further, the memory storage performed by one distinct memory portion as described above may be performed by two memory portions.

Further, various technologies may be used to provide communication between the various processors and/or memories, as well as to allow the processors and/or the memories to communicate with any other entity; i.e., so as to obtain further instructions or to access and use remote memory stores, for example. Such technologies used to provide such communication might include a network, the Internet, Intranet, Extranet, a LAN, an Ethernet, wireless communication via cell tower or satellite, or any client server system that provides communication, for example. Such communications technologies may use any suitable protocol such as TCP/IP, UDP, or OSI, for example.

As described above, a set of instructions may be used in the processing of embodiments. The set of instructions may be in the form of a program or software. The software may be in the form of system software or application software, for example. The software might also be in the form of a collection of separate programs, a program module within a larger program, or a portion of a program module, for example. The software used might also include modular programming in the form of object-oriented programming. The software tells the processing machine what to do with the data being processed.

Further, it is appreciated that the instructions or set of instructions used in the implementation and operation of embodiments may be in a suitable form such that the processing machine may read the instructions. For example, the instructions that form a program may be in the form of a suitable programming language, which is converted to machine language or object code to allow the processor or processors to read the instructions. That is, written lines of programming code or source code, in a particular programming language, are converted to machine language using a compiler, assembler or interpreter. The machine language is binary coded machine instructions that are specific to a particular type of processing machine, i.e., to a particular type of computer, for example. The computer understands the machine language.

Any suitable programming language may be used in accordance with the various embodiments. Also, the instructions and/or data used in the practice of embodiments may utilize any compression or encryption technique or algorithm, as may be desired. An encryption module might be used to encrypt data. Further, files or other data may be decrypted using a suitable decryption module, for example.

As described above, the embodiments may illustratively be embodied in the form of a processing machine, including a computer or computer system, for example, that includes at least one memory. It is to be appreciated that the set of instructions, i.e., the software for example, that enables the computer operating system to perform the operations described above may be contained on any of a wide variety of media or medium, as desired. Further, the data that is processed by the set of instructions might also be contained on any of a wide variety of media or medium. That is, the particular medium, i.e., the memory in the processing machine, utilized to hold the set of instructions and/or the data used in embodiments may take on any of a variety of physical forms or transmissions, for example. Illustratively, the medium may be in the form of a compact disc, a DVD, an integrated circuit, a hard disk, a floppy disk, an optical disc, a magnetic tape, a RAM, a ROM, a PROM, an EPROM, a wire, a cable, a fiber, a communications channel, a satellite transmission, a memory card, a SIM card, or other remote transmission, as well as any other medium or source of data that may be read by the processors.

Further, the memory or memories used in the processing machine that implements embodiments may be in any of a wide variety of forms to allow the memory to hold instructions, data, or other information, as is desired. Thus, the memory might be in the form of a database to hold data. The database might use any desired arrangement of files such as a flat file arrangement or a relational database arrangement, for example.

In the systems and methods, a variety of “user interfaces” may be utilized to allow a user to interface with the processing machine or machines that are used to implement embodiments. As used herein, a user interface includes any hardware, software, or combination of hardware and software used by the processing machine that allows a user to interact with the processing machine. A user interface may be in the form of a dialogue screen for example. A user interface may also include any of a mouse, touch screen, keyboard, keypad, voice reader, voice recognizer, dialogue screen, menu box, list, checkbox, toggle switch, a pushbutton or any other device that allows a user to receive information regarding the operation of the processing machine as it processes a set of instructions and/or provides the processing machine with information. Accordingly, the user interface is any device that provides communication between a user and a processing machine. The information provided by the user to the processing machine through the user interface may be in the form of a command, a selection of data, or some other input, for example.

As discussed above, a user interface is utilized by the processing machine that performs a set of instructions such that the processing machine processes data for a user. The user interface is typically used by the processing machine for interacting with a user either to convey information or receive information from the user. However, it should be appreciated that in accordance with some embodiments of the system and method, it is not necessary that a human user actually interact with a user interface used by the processing machine. Rather, it is also contemplated that the user interface might interact, i.e., convey and receive information, with another processing machine, rather than a human user. Accordingly, the other processing machine might be characterized as a user. Further, it is contemplated that a user interface utilized in the system and method may interact partially with another processing machine or processing machines, while also interacting partially with a human user.

It will be readily understood by those persons skilled in the art that embodiments are susceptible to broad utility and application. Many embodiments and adaptations of the present invention other than those herein described, as well as many variations, modifications and equivalent arrangements, will be apparent from or reasonably suggested by the foregoing description thereof, without departing from the substance or scope.

Accordingly, while the embodiments of the present invention have been described here in detail in relation to its exemplary embodiments, it is to be understood that this disclosure is only illustrative and exemplary of the present invention and is made to provide an enabling disclosure of the invention. Accordingly, the foregoing disclosure is not intended to be construed or to limit the present invention or otherwise to exclude any other such embodiments, adaptations, variations, modifications, or equivalent arrangements.

What is claimed is:

1. A method, comprising:
receiving, by a computer program executed by a backend electronic device, a communication a customer regarding a mobile application;
retrieving, by the computer program, customer information associated with the customer;
identifying, by the computer program, a potential issue with the mobile application based on the customer information;
generating, by the computer program, mock data for the customer;
generating, by the computer program, a simulated customer profile from the mock data and the customer information;
simulating, by the computer program, execution of the mobile application using a mobile application simulator for the mobile application using the simulated customer profile;
troubleshooting, by the computer program, the potential issue using the mobile application simulator; and
sending, by the computer program, a control signal to the mobile application to address the potential issue.

2. The method of claim 1, wherein the customer information comprises account information, mobile application feature information, and customer preferences for the mobile application.

3. The method of claim 1, wherein the potential issue with the mobile application is identified using a rules based engine.

4. The method of claim 1, wherein the potential issue with the mobile application is identified using a database of known issues.

5. The method of claim 1, wherein the potential issue with the mobile application is identified using a machine learning model trained on historical support cases.

6. The method of claim 1, wherein the mock data is generated based on predefined templates for the potential issue.

7. The method of claim 1, wherein the mock data comprises plausible merchant names, plausible transaction amounts, and/or plausible error messages.

8. The method of claim 1, wherein the mock data is generated using a large language models.

9. The method of claim 1, wherein the control signal causes the mobile application to turn a feature on or off.

10. The method of claim 1, wherein the control signal causes the mobile application to display a page.

11. A non-transitory computer readable storage medium, including instructions stored thereon, which when read and executed by one or more computer processors, cause the one or more computer processors to perform steps comprising:
receiving a communication a customer regarding a mobile application;
retrieving customer information associated with the customer;
identifying a potential issue with the mobile application based on the customer information;
generating mock data for the customer;
generating a simulated customer profile from the mock data and the customer information;
simulating execution of the mobile application using a mobile application simulator for the mobile application using the simulated customer profile;
troubleshooting the potential issue using the mobile application simulator; and
sending a control signal to the mobile application to address the potential issue.

12. The non-transitory computer readable storage medium of claim 11, wherein the customer information comprises account information, mobile application feature information, and customer preferences for the mobile application.

13. The non-transitory computer readable storage medium of claim 11, wherein the potential issue with the mobile application is identified using a rules based engine.

14. The non-transitory computer readable storage medium of claim 11, wherein the potential issue with the mobile application is identified using a database of known issues.

15. The non-transitory computer readable storage medium of claim 11, wherein the potential issue with the mobile application is identified using a machine learning model trained on historical support cases.

16. The non-transitory computer readable storage medium of claim 11, wherein the mock data is generated based on predefined templates for the potential issue.

17. The no-transitory computer readable storage medium of claim 11, wherein the mock data comprises plausible merchant names, plausible transaction amounts, and/or plausible error messages.

18. The non-transitory computer readable storage medium of claim 11, wherein the mock data is generated using a large language models.

19. The non-transitory computer readable storage medium of claim 11, wherein the control signal causes the mobile application to turn a feature on or off.

20. The non-transitory computer readable storage medium of claim 11, wherein the control signal causes the mobile application to display a page.
